SEQUENCE LISTING (8 sequences)

SEQ ID NO: 1 - protein, 31 aa, Chitinophaga sancti
QEQAQTYLDGYNKTYQDLVYKDNLAQWTLNT

SEQ ID NO: 2 - protein, 32 aa, Nipponia nippon
VTQQAQMFLEEFNRRAENISYESSLASWDYNT

SEQ ID NO: 3 - protein, 31 aa, Mus musculus
EENAKTFLNNFNQEAEDLSYQSSLASWNYNT

SEQ ID NO: 4 - protein, 32 aa, Perca fluviatilis
VENKANEFLQKFDEEATRRMYQYSLASWAYNT

SEQ ID NO: 5 - protein, 31 aa, Chitinophaga sp.
RQQAQTYLDSYNKTYQDLIYKDNLAQWTLNT

SEQ ID NO: 6 - protein, 32 aa, Anolis carolinensis
VTQQAAEFLLQFNINAENRSYESSLASWDYNT

SEQ ID NO: 7 - protein, 32 aa, Alligator mississippiensis
VPQNVTTFLNQFNQNAEGLYYESSLASWAYNT

SEQ ID NO: 8 - protein, 32 aa, Homo sapiens
IEEQAKTFLDKFNHEAEDLFYQSSLASWNYNT

(19) United States
(12) Patent Application Publication
(10) Pub. No.: US 2026/0167674 A1
(43) Pub. Date: Jun. 18, 2026

(54) ACE2 HOMOLOGOUS PEPTIDE SEQUENCES

(71) Applicant: BEZMIALEM VAKIF ÜNIVERSITESI, Istanbul (TR)

(72) Inventors: SERDAR UYSAL, Istanbul (TR); MOHAMMAD ASIF KHAN, Istanbul (TR)

(73) Assignee: BEZMIALEM VAKIF ÜNIVERSITESI, Istanbul (TR)

(21) Appl. No.: 18/845,882

(22) PCT Filed: Apr. 22, 2024
(86) PCT No.: PCT/TR2024/050405

§ 371 Date: Sep. 11, 2024

(30) Foreign Application Priority Data
Apr. 26, 2023 (TR) 2023/004618

(51) Int. Cl. C07K 14/005; A61K 38/16; A61K 39/00; A61K 39/39; C07K 7/08; C12N 7/00

(52) U.S. Cl. CPC C07K 14/005; A61K 38/16; A61K 39/39; C07K 7/08; C12N 7/00; A61K 2039/505; C07K 2317/56; C07K 2317/565

(57) ABSTRACT

Peptide sequences that are ACE2 homologues are provided. Compared to the wild-type ACE2 in the host, the peptide sequences bind with higher affinity to the receptor-binding domain (RBD), thus inhibiting this interaction by competitively inhibiting the binding of the virus RBD region of SARS-COV-2 with the human ACE2. The peptide sequences can be included in a pharmaceutical composition.

### TECHNICAL FIELD

The invention discloses peptide sequences that are ACE2 homologues. Compared to the wild-type ACE2 in the host, the peptide sequences of the invention bind with higher affinity to the receptor-binding domain (RBD), thus inhibiting the interaction by competitively inhibiting the binding of the RBD region to the of SARS-COV-2 with ACE2 in host cells.

### STATE OF THE ART

With the emergence of the COVID-19 disease caused by the SARS-COV-2 virus at the end of 2019, the virus spread worldwide in a short-time and negatively affected the health of millions of people. Based on the World Health Organization data, the number of cases exceeded 609 million, and deaths exceeded 6.5 million, as of September 2022.

For the SARS-COV-2 virus to initiate infection, the surface protein of the virus termed as spike, through its receptor-binding domain (RBD) needs to bind to ACE2, a membrane protein responsible for the maturation of angiotensin that controls blood pressure in the human respiratory system. Prevention of RBD-ACE2 interaction will prevent the entry of the virus into host cells and the spread of the disease.

In the prior art, Chevalier et al. developed mini-proteins and carried out their characterizations with comprehensive computational design studies against H1 hemagglutinin against influenza A. After allowing a large number of visually designed mini-proteins to form complexes with the target molecule, they examined the regions forming the interface, binding affinity, and stability. They synthesized the mini-protein sequences selected for characterization and transformed them into yeast cells to create a library in the yeast representation method. The mini-proteins were expressed on the surface of yeast cells with fluorescence-marked targets and those with high affinity were selected. They observed that selected mini-proteins provided protection against repeated doses of influenza.

In another similar study, Cao et al. used de novo and docking approaches to determine small peptide sequences that could be administered intranasally to prevent virus interaction with the human ACE2 protein by interacting with certain regions that bind with the ACE2 helix (23-46 amino acids) and the receptor-binding domain (RBD) against the SARS-COV-2 virus. They selected and characterized those with high affinity. The SARS-COV-2 RBD was marked as fluorescence from the large yeast representation library they established with the peptides they designed. They stated that the selected small peptide sequences inhibited binding with ACE2 and had advantages in terms of recombinant production, stability, and case of administration to patients with high affinity and low molecular weight compared to antibodies used for similar purposes.

Unlike the above methods, high-affinity nanobodies that complex with RBD were developed through immunization with recombinant RBD to prevent RBD interaction with ACE2.

### FIGURES

FIG. 1: ACE2 Homo sapiens reference peptide in complex with SARS-COV-2 RBD; NP_001373189.1.

FIG. 2: peptide2 in complex with SARS-COV-2 RBD. WP_072363071.1:30-60 M2 family metallopeptidase [Chitinophaga sancti].

FIG. 3: peptide3 in complex with SARS-COV-2 RBD. KFQ92425.1:20-51 Angiotensin-converting enzyme 2 [Nipponia nippon].

FIG. 4: peptide4 in complex with SARS-COV-2 RBD. BAB40431.1:22-52 angiotensin-converting enzyme-related carboxypeptidase [Mus musculus].

FIG. 5: peptide5 in complex with SARS-COV-2 RBD. KAF1392660.1:22-53 hypothetical protein PFLUV_G00030370 [Perca fluviatilis].

FIG. 6: peptide6 in complex with SARS-COV-2 RBD. WP_116857206.1:30-60 M2 family metallopeptidase [Chitinophaga sp. K20C18050901].

FIG. 7: peptide7 in complex with SARS-COV-2 RBD. XP_008105455.1:29-60 PREDICTED: angiotensin-converting enzyme 2 [Anolis carolinensis].

FIG. 8: peptide8 in complex with SARS-COV-2 RBD. XP_019350687.1:16-47 PREDICTED: angiotensin-converting enzyme 2 [Alligator mississippiensis]. P

FIG. 9: Binding activity of Peptide #1(Ref.) (A), Peptide #2 (B), Peptide #3 (C) and Peptide #4 (D)

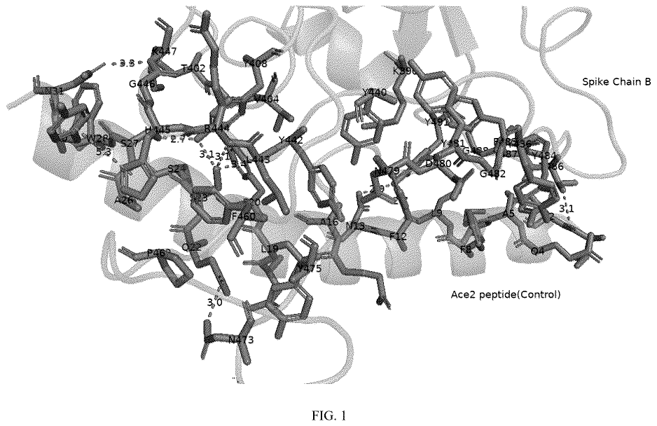

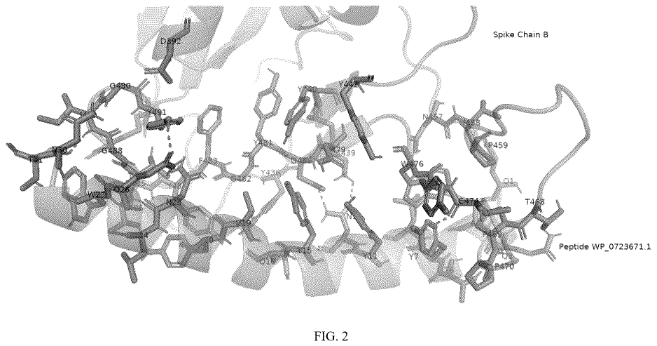

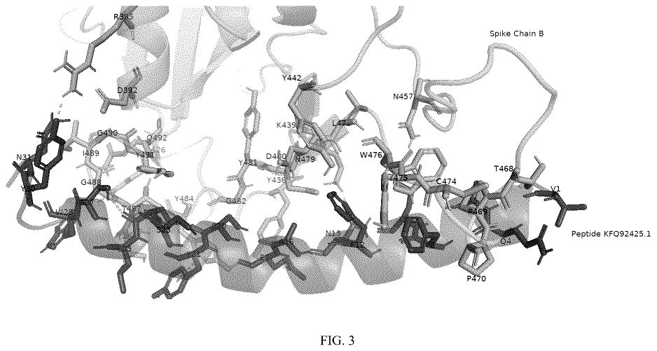

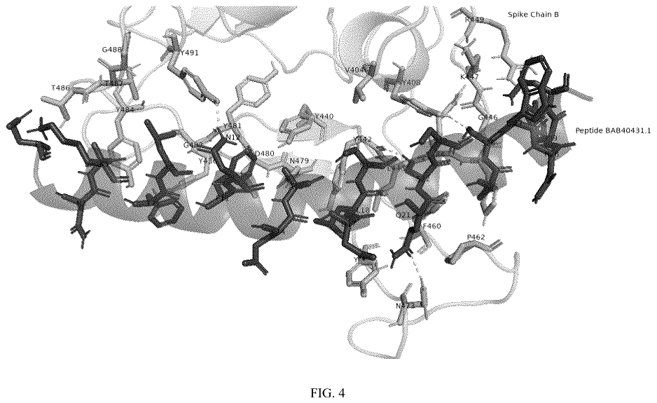

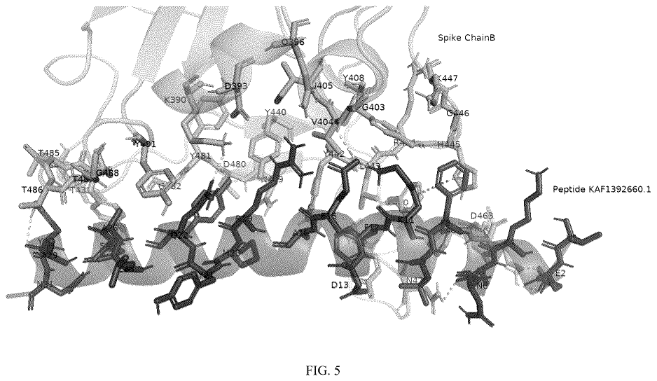

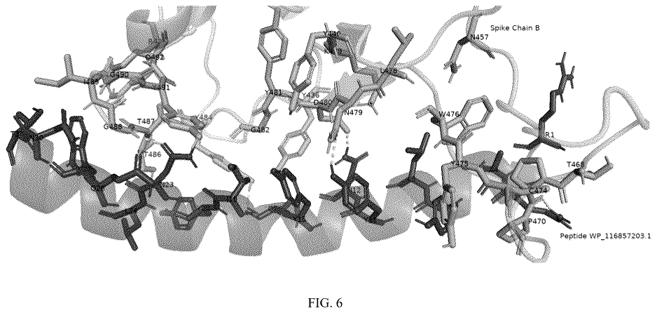

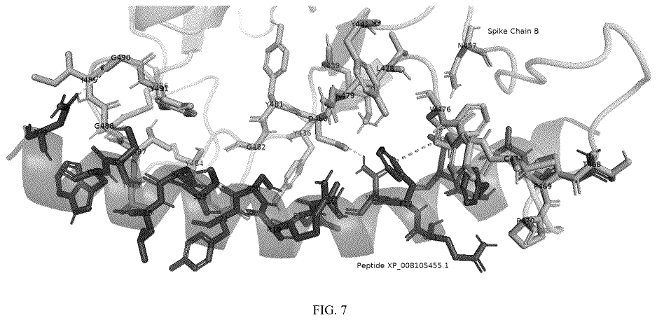

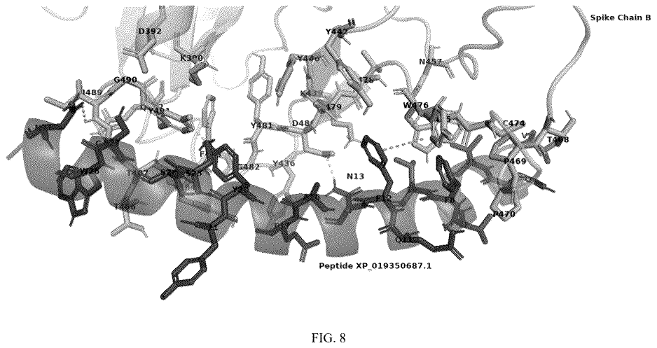

### BRIEF DESCRIPTION OF THE INVENTION

Compared to the wild-type ACE2 in the host, the peptide sequences, which are the ACE2 homologues of the invention, bind with higher affinity with the receptor-binding domain (RBD) of the virus, thus competitively inhibiting the binding of the RBD region of SARS-CoV-2 with ACE2 in host cells and inhibiting this interaction.

### DETAILED DESCRIPTION OF THE INVENTION

There is a need for a method that can be developed quickly against viral antigenic determinants in viral diseases that spread rapidly, affect the whole world, and need to be controlled urgently, such as during a pandemic. Thus, the spread of highly pathogenic viruses that can switch between species, such as SARS-COV-2, need to be prevented at the earliest. ACE2 homologous peptides of the invention show high binding affinity to SARS-COV-2 RBD, and their affinity is higher than human ACE2 receptor protein. These peptides are means to competitively inhibit the binding of the virus RBD to with the human ACE2 receptor protein.

While determining the peptide sequences of the invention, the information that SARS-COV-2 needs to interact with the ACE2 receptor in host cells in the human respiratory system to initiate infection was used. Accordingly, BLAST search was applied with the sequence of human ACE2 receptor protein of consisting 32 amino acids (21-IEEQAKTFLDKFNHEAEDLFYQSSLASWNYNT-52), which interacts with RBD, 624 hits, comprising of different ACE2 homologous peptides of different species in nature, were selected to act as a competitive ligand to human ACE2 receptor protein for RBD.

Complexes were modeled separately for the 624 different homologous ACE2 peptides with the SARS-COV-2 spike protein, which was carried out in three dimensions by use of HADDOCK 2.4, a web-based molecular docking approach. When the complexes were examined, the mean binding affinity (Kd) of the peptides at 37° C. and the mean binding free energy (AG) were compared with the human ACE2 protein (peptide1), which was taken as reference, by use of the prodigy web tool. At the end of the evaluation, among the peptides, the following were selected; Chitinophaga sancti (bacteria), Nipponia nippon (ibis), Mus musculus (mouse), Perca fluviatilis (perch), Chitinophaga sp. K20C18050901 (bacteria), and Anolis carolinensis (anole), and Alligator mississippiensis (alligator). The complexes were also examined using the PyMOL tool for the best binding affinities and the number of polar interactions between them and the spike protein, which was listed in terms of the number of amino acids in the interface of the complex they formed, and based on all these evaluations, three homologous peptide sequences peptide2 of Chitinophaga sancti species, peptide3 of Nipponia nippon species and peptide4 of Mus musculus species). Synthesis of the three determined homologous peptide sequences was carried out and the binding affinity of the peptides with the SARS-COV-2 RBD protein, purified by recombinant expression in Pichia pastoris, was determined by surface plasmon resonance method. As a result, the three selected ACE2 homologous peptides showed binding affinity with RBD at a higher rate than predicted by the molecular docking approach. Accordingly, the binding affinities determined are 318-441 picomolar (pM) for the human ACE2 protein (peptide1) taken as reference, 3.55-4.09 μM for peptide2, 0.59-1.65 μM for peptide3 and 1.26-8.16 μM for peptide4. Therefore, when compared with the reference human ACE2 protein, binding affinities were found to be 89-107 times higher for peptide2 (Chitinophaga sancti), 538-267 times higher for peptide3 (Nipponia nippon) and 252-54 times higher for peptide4 (Mus musculus).

peptide1:
IEEQAKTFLDKFNHEAEDLFYQSSLASWNYNT
(ACE2 Homo sapiens Reference; NP_001373189.1) (peptidel is the reference human
ACE2 protein, prior art)
peptide2:
(SEQ NO 1)
QEQAQTYLDGYNKTYQDLVYKDNLAQWTLNT
(WP_072363071.1: 30-60 M2 family metallopeptidase [Chitinophaga sancti])
peptide3:
(SEQ NO 2)
VTQQAQMFLEEFNRRAENISYESSLASWDYNT
(KFQ92425.1: 20-51 Angiotensin-converting enzyme 2 [Nipponia nippon])
peptide4:
(SEQ NO 3)
EENAKTFLNNFNQEAEDLSYQSSLASWNYNT
(BAB40431.1: 22-52 angiotensin-converting enzyme-related carboxypeptidase [Mus
musculus])
peptide5:
(SEQ NO 4)
VENKANEFLQKFDEEATRRMYQYSLASWAYNT
(KAF1392660.1: 22-53 hypothetical protein PFLUV_G00030370 [Perca fluviatilis])
peptide6:
(SEQ NO 5)
RQQAQTYLDSYNKTYQDLIYKDNLAQWTLNT
(WP_116857206.1: 30-60 M2 family metallopeptidase [Chitinophaga sp. K20C18050901])
peptide7:
(SEQ NO 6)
VTQQAAEFLLQFNINAENRSYESSLASWDYNT
(XP 008105455.1: 29-60 PREDICTED: angiotensin-converting enzyme 2 [Anolis
carolinensis])
peptide8:
(SEQ NO 7)
VPQNVTTFLNQFNQNAEGLYYESSLASWAYNT
(XP 019350687.1: 16-47 PREDICTED: angiotensin-converting enzyme 2 [Alligator
mississippiensis])

The 3-letter representations of the peptide sequences are also presented below.

Peptide 1:
Ile Glu Glu Gln Ala Lys Thr Phe Leu Asp Lys Phe
Asn His Glu Ala Glu Asp Leu Phe Tyr
Gln Ser Ser Leu Ala Ser Trp Asn Tyr Asn Thr
Peptide 2:
Gln Glu Gln Ala Gln Thr Tyr Leu Asp Gly Tyr Asn
Lys Thr Tyr Gln Asp Leu Val Tyr Lys
Asp Asn Leu Ala Gln Trp Thr Leu Asn Thr
Peptide 3:
Val Thr Gln Gln Ala Gln Met Phe Leu Glu Glu Phe
Asn Arg Arg Ala Glu Asn Ile Ser Tyr
Glu Ser Ser Leu Ala Ser Trp Asp Tyr Asn Thr
Peptide 4:
Glu Glu Asn Ala Lys Thr Phe Leu Asn Asn Phe Asn
Gln Glu Ala Glu Asp Leu Ser Tyr Gln
Ser Ser Leu Ala Ser Trp Asn Tyr Asn Thr
Peptide 5:
Val Glu Asn Lys Ala Asn Glu Phe Leu Gln Lys Phe
Asp Glu Glu Ala Thr Arg Arg Met Tyr
Gln Tyr Ser Leu Ala Ser Trp Ala Tyr Asn Thr
Peptide 6:
Arg Gln Gln Ala Gln Thr Tyr Leu Asp Ser Tyr Asn
Lys Thr Tyr Gln Asp Leu Ile Tyr Lys
Asp Asn Leu Ala Gln Trp Thr Leu Asn Thr
Peptide 7:
Val Thr Gln Gln Ala Ala Glu Phe Leu Leu Gln Phe
Asn Ile Asn Ala Glu Asn Arg Ser Tyr
Glu Ser Ser Leu Ala Ser Trp Asp Tyr Asn Thr
Peptide 8:
Val Pro Gln Asn Val Thr Thr Phe Leu Asn Gln Phe
Asn Gln Asn Ala Glu Gly Leu Tyr Tyr
Glu Ser Ser Leu Ala Ser Trp Ala Tyr Asn Thr

The affinity of the peptides (peptide2, peptide3, peptide4) of the invention designed with a computational approach and selected from different ACE2 homologous species sequences against the SARS-COV-2 RBD region is higher than the affinity of the human ACE2 peptide against the SARS-COV-2 RBD region.

In the detection and characterization of the peptides (peptide2, peptide3, peptide4) subject to the invention, a high throughput virtual screening approach was used comprehensively using the modeling and other tools mentioned above, and then experimental laboratory applications were used.

Estimated binding affinity (equilibrium dissociation constant, Kd) and estimated binding free energy (ΔG) data of the complexes formed by 624 peptides with S protein, which were analyzed by binding conformation with SARS-COV-2 S protein model with the web-based HADDOCK 2.4 molecular docking tool, were obtained by using a prodigy web server, set at a temperature of 37° C. ACE2 Homo sapiens peptide was used as positive control, and the peptides with the best binding affinity were selected with the Kd and ΔG data, relative to the positive control. With the analyses made, complexes that met the desired parameters were selected. Interface residues of molecular complexes were visualised by use of PyMol tool (The PyMOL Molecular Graphics System, Version 2.0 Schrödinger, LLC) and PyMol script. Accordingly, when ranked in terms of predicted binding affinity, predicted binding free energy, number of polar interactions, and percentage of interface residue, seven peptides, excluding the reference peptide (peptide1), were determined. peptide2, peptide3, and peptide4, which are the top 3 among them, were synthesized and binding affinity was determined with SARS-COV-2 RBD expressed and purified in P. pastoris in vitro.

Table 1 shows the top seven ACE2 homologous peptides in terms of predicted binding affinity, predicted binding free energy, number of polar interactions and percentage of interface residue among the peptides obtained using the high throughput virtual screening approach, including the Homo sapiens ACE2 reference. Of these, the first four peptides were synthesized, and their binding affinities were confirmed experimentally.

TABLE 1

| | | | Estimated | Estimated | | |
|---|---|---|---|---|---|---|
| | | | binding | binding free | Number | |
| | | | affinity | energy | of | |
| | | | Kd (nM) | (ΔG | polar | Interface |
| | | | at 37.0° | (kcal/mol) | inter- | residues |
| | Peptide | Description | C.) | at 37.0° C.) | actions | (%) |
| #1 | IEEQAKTFLDKF | ACE2 Homo sapiens | 27.00 | −10.7 | 15 | 65.60 |
| Ref. | NHEAEDLFYQSS | Reference; | | | | |
| | LASWNYNT | NP_001373189.1 | | | | |
| #2 | QEQAQTYLDGY | WP_072363071.1: | 0.17 | −13.9 | 11 | 64.50 |
| | NKTYQDLVYKD | 30-60 M2 family | | | | |
| | NLAQWTLNT | metallopeptidase | | | | |
| | | [Chitinophaga sancti] | | | | |
| #3 | VTQQAQMFLEEF | KFQ92425.1: 20-51 | 0.14 | −14.0 | 10 | 62.50 |
| | NRRAENISYESSL | Angiotensin- | | | | |
| | ASWDYNT | converting enzyme 2 | | | | |
| | | [Nipponia nippon] | | | | |
| #4 | EENAKTFLNNFN | BAB40431.1: 22-52 | 0.16 | −13.9 | 9 | 64.51 |
| | QEAEDLSYQSSL | angiotensin-converting | | | | |
| | ASWNYNT | enzyme-related | | | | |
| | | carboxypeptidase | | | | |
| | | [Mus musculus] | | | | |
| #5 | VENKANEFLQKF | KAF1392660.1: 22-53 | 0.18 | −13.8 | 9 | 59.38 |
| | DEEATRRMYQY | hypothetical protein | | | | |
| | SLASWAYNT | PFLUV_G00030370 | | | | |
| | | [Perca fluviatilis] | | | | |
| #6 | RQQAQTYLDSY | WP_116857206.1: | 0.18 | −13.8 | 7 | 58.06 |
| | NKTYQDLIYKDN | 30-60 M2 family | | | | |
| | LAQWTLNT | metallopeptidase | | | | |
| | | [Chitinophaga sp. | | | | |
| | | K20C18050901] | | | | |
| #7 | VTQQAAEFLLQF | XP_008105455.1: | 0.18 | −13.8 | 5 | 56.25 |
| | NINAENRSYESSL | 29-60 PREDICTED: | | | | |
| | ASWDYNT | angiotensin-converting | | | | |
| | | enzyme 2 [Anolis | | | | |
| | | carolinensis] | | | | |
| #8 | VPQNVTTFLNQF | XP_019350687.1: | 0.18 | — | 6 | 56.25 |
| | NQNAEGLYYESS | 16-47 PREDICTED: | | | | |
| | LASWAYNT | angiotensin-converting | | | | |
| | | enzyme 2 [Alligator | | | | |
| | | mississippiensis] | | | | |

Table 2 (reference) shows surface plasmon resonance (SPR) results for peptide1, peptide2, peptide3, and peptide4 showing binding affinity with SARS-COV-2 RBD produced by expression in Pichia pastoris.

TABLE 2

| | Kd (pM) at 25° C. |
|---|---|
| Peptide #1 | 318.00-441.00 |
| (Reference) | |
| Peptide #2 | 3.55-4.09 |
| Peptide #3 | 0.59-1.65 |
| Peptide #4 | 1.26-8.16 |

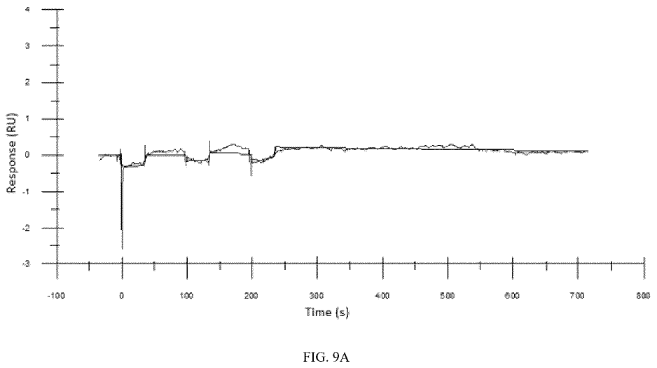

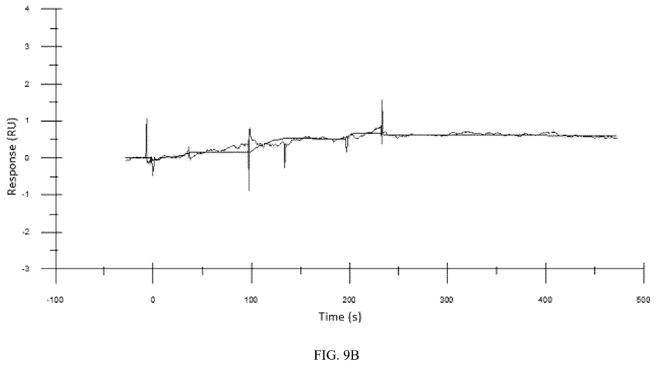

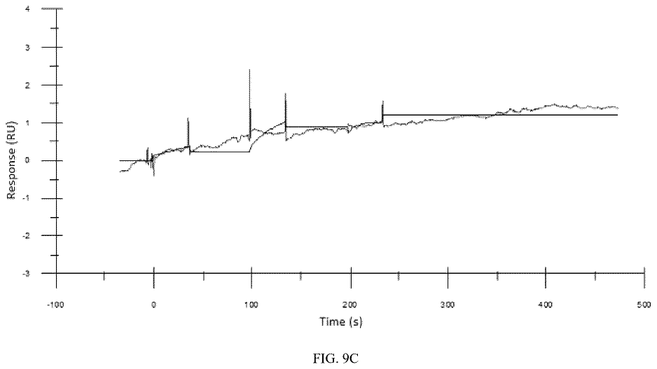

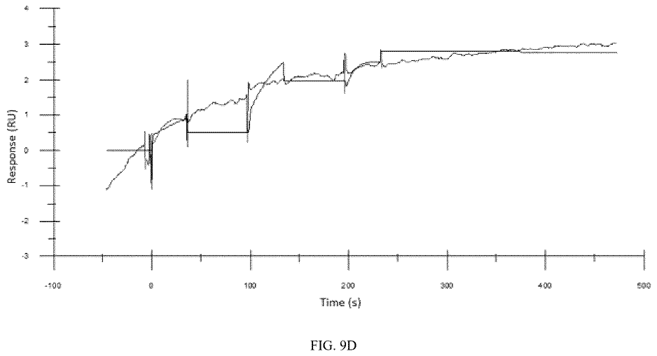

In FIG. 9, SARS-COV-2 is examined against RBD. With anti-His tag immobilized to the flow cell surface, SARS-COV-2 with His-tag was captured on the surface of the RBD flow cell. The peptides were then flowed through the flow cell surface to provide SARS-COV-2 RBD binding on the surface. Kinetic parameters show an interaction between peptides and RBD-His protein.

What is claimed is:

1. An angiotensin converting enzyme 2 (ACE2) homologous peptide, comprising one of the following sequences:
(SEQ ID NO: 1)
QEQAQTYLDGYNKTYQDLVYKDNLAQWTLNT
(SEQ ID NO: 2)
VTQQAQMFLEEFNRRAENISYESSLASWDYNT
(SEQ ID NO: 3)
EENAKTFLNNFNQEAEDLSYQSSLASWNYNT
(SEQ ID NO: 4)
VENKANEFLQKFDEEATRRMYQYSLASWAYNT
(SEQ ID NO: 5)
RQQAQTYLDSYNKTYQDLIYKDNLAQWTLNT
(SEQ ID NO: 6)
VTQQAAEFLLQFNINAENRSYESSLASWDYNT
(SEQ ID NO: 7)
VPQNVTTFLNQFNQNAEGLYYESSLASWAYNT

2. The ACE2 homologous peptide according to claim 1, comprising the following sequence:
(SEQ ID NO: 1)
QEQAQTYLDGYNKTYQDLVYKDNLAQWTLNT.

3. The ACE2 homologous peptide according to claim 1, comprising the following sequence:
(SEQ ID NO: 2)
VTQQAQMFLEEFNRRAENISYESSLASWDYNT.

4. The ACE2 homologous peptide according to claim 1, comprising the following sequence:
(SEQ ID NO: 3)
EENAKTFLNNFNQEAEDLSYQSSLASWNYNT.

5. The ACE2 homologous peptide according to claim 1, comprising the following sequence:
(SEQ ID NO: 4)
VENKANEFLQKFDEEATRRMYQYSLASWAYNT.

6. The ACE2 homologous peptide according to claim 1, comprising the following sequence:
(SEQ ID NO: 5)
RQQAQTYLDSYNKTYQDLIYKDNLAQWTLNT.

7. The ACE2 homologous peptide according to claim 1, comprising the following sequence:
(SEQ ID NO: 6)
VTQQAAEFLLQFNINAENRSYESSLASWDYNT.

8. The ACE2 homologous peptide according to claim 1, comprising the following sequence:
(SEQ ID NO: 7)
VPQNVTTFLNQFNQNAEGLYYESSLASWAYNT.

9. A pharmaceutical composition, comprising the ACE2 homologous peptide according to claim 1, wherein the pharmaceutical composition competitively inhibits a binding of a receptor-binding domain region of severe acute respiratory syndrome coronavirus 2 (SARS-COV-2) with ACE2 in host cells.
